(19) United States
(12) Patent Application Publication
(10) Pub. No.: US 2025/0019356 A1
(43) Pub. Date: Jan. 16, 2025

(54) OLEFIN EPOXIDATION WITH MODERATOR MANAGEMENT

(71) Applicant: Scientific Design LLC, Little Ferry, NJ (US)

(72) Inventors: Xiankuan Zhang, Houston, TX (US); Andrew D. Schmitz, Hackensack, NJ (US)

(73) Assignee: Scientific Design LLC, Little Ferry, NJ (US)

(21) Appl. No.: 18/767,466

(22) Filed: Jul. 9, 2024

Related U.S. Application Data
(60) Provisional application No. 63/526,833, filed on Jul. 14, 2023.

(51) Int. Cl. C07D 301/36; B01J 21/04; B01J 23/66

(52) U.S. Cl. CPC C07D 301/36; B01J 21/04; B01J 23/66

(57) ABSTRACT

A moderator management process that can be employed during an epoxidation process is disclosed. The disclosed moderator management process provides optimum catalyst performance without having to rely on a trial and error or using elaborate equations as disclosed in the prior art. In the disclosed moderator management process, the optimum halide-containing moderator concentrations can be determined by maintaining ΔEa from 30 kJ/mol to 300 kJ/mol, wherein ΔEa is the difference in activation energies between the reaction to remove halide from the surface of the epoxidation catalyst and the reaction to deposit halide on the surface of the epoxidation catalyst.

### FIELD OF THE DISCLOSURE

The present disclosure claims benefit of U.S. Provisional Application Ser. No. 63/526,833 filed Jul. 14, 2023, the entire content and disclosure of which is incorporated herein by reference.

The present disclosure relates to the epoxidation of an olefin, specifically ethylene, to an olefin oxide, especially ethylene oxide (EO), in which an epoxidation catalyst is employed. More particularly, the present disclosure relates to an epoxidation process in which moderator management is employed to provide optimum catalyst performance at a given reaction temperature.

### BACKGROUND

The catalytic epoxidation of an olefin in the presence of a silver-based catalyst producing an olefin oxide is well known in the art. The catalytic oxidation of ethylene to ethylene oxide is usually practiced as a gas-phase process in which the feed is contacted in the gas phase with the catalyst present as a solid material. The catalyst is typically positioned in a tubular, packed-bed reactor, and the reactor is typically equipped with heat exchange facilities to heat or cool the catalyst. The temperature of the process can be measured either by inserting thermocouples into reactor tubes in contact with the catalyst, which is commonly referred as the catalyst bed temperature, or determined by measuring the reactor coolant temperature, which is the temperature of the coolant contained in the reactor shell, outside the reactor tubes.

Halogen-containing compounds, especially chlorohydrocarbons, have long been used in the feed mixture for the gas phase production of ethylene oxide (see e.g., U.S. Pat. No. 2,279,469; U.K. Patent No. 1,055,147, and EPO Patent No. 0 352 850 B1). The added halogen-containing compound has been variously known as an inhibitor, modifier, moderator, anti-catalyst, and promoter, and is herein called a moderator. The moderator plays a key role in maintaining the catalyst's activity and selectivity for producing ethylene oxide. This is especially true for rhenium-containing. highly selective catalysts where optimum performance can only be obtained within a narrow moderator concentration range within the feed mixture. Furthermore, this optimum moderator concentration range is not fixed, but it changes with temperature of the process. Catalyst performance deteriorates with time, so temperature is generally increased with time to maintain a constant rate of ethylene oxide production. The moderator concentration must therefore be incrementally increased with temperature to keep the catalyst operating at peak efficiency.

A catalyst's efficiency for producing ethylene oxide in catalytic oxidation of ethylene is very important. This efficiency is a combination of catalyst selectivity and catalyst activity. Selectivity is defined as the amount of ethylene oxide produced for a given amount of ethylene reacted on the catalyst; whereas, the activity is customarily expressed in terms of the catalyst bed temperature, or the reactor coolant temperature required for production of ethylene oxide at a given rate. The rate of ethylene oxide production is commonly expressed in terms of the amount of ethylene oxide produced per unit volume (or mass) of the catalyst per unit time. Because the selectivity and activity of the highly selective catalyst are both very sensitive to the moderator concentration in the reactor feed, the moderator concentration must be carefully tuned to maximize the efficiency of the catalyst.

Historically, operators of the highly selective catalyst have attempted to optimize the moderator concentration by trial and error. In such instances, the skilled operator would make an incremental change to the moderator concentration, up or down, and then wait to see the change in catalyst efficiency. If catalyst efficiency improved, then the operator would continue making incremental changes in the same direction until maximum ethylene oxide selectivity could be obtained at the lowest reactor coolant temperature. If catalyst efficiency had not improved with the change in moderator concentration, then the operator had to reverse the steps and attempt to optimize catalyst efficiency by moving moderator concentration in the opposite direction. This trial-and-error optimization process is painstakingly slow and tedious and generally must be executed by someone skilled in the art of operating the highly selective catalyst. The trial-and-error optimization process can be especially difficult or impossible if the temperature of the process is fluctuating.

Increasing the moderator concentration above the optimum generally causes selectivity to decrease, but because the catalyst function may degrade more quickly at higher temperatures, it is sometimes desirable to increase the moderator concentration still further, anyway, and to suffer some selectivity loss in exchange for operating the catalyst at a lower temperature. Temperature can increase or decrease when a change is made to the operating conditions of the catalytic oxidation of ethylene to ethylene oxide process, Temperature is generally increased over the service life of the catalyst to compensate for the loss in the catalyst's activity. Irrespective of the cause, the moderator concentration must be re-optimized every time the temperature changes. Again, this means making small adjustments to the moderator concentration until it appears that maximum catalyst operating efficiency has been reestablished. Even for persons skilled in the art, these repetitive, incremental re-optimizations are difficult and make it inherently difficult to keep the catalyst operating at peak efficiency and likewise, to maintain high efficiency in the overall catalytic process.

Techniques for such automation of the moderator levels have been proposed in the prior art. For example, U.S. Pat. Nos. 7,657,331 and 7,657,332 recite specific formulas and ratios to predict what the optimal modifier (herein, moderator) levels should be, making use of a Q value for calculating the correct modifier concentration. The value of Q value is described as the ratio of the total effective modifier to the total effective hydrocarbon. The effective hydrocarbon value is determined by multiplying the molar concentration for each species of hydrocarbon by a correction factor that (according to theory) accounts for the differences in the ability of the different hydrocarbons to remove/strip reaction modifier from the surface of the catalyst; whereas, the effective modifier value is determined by multiplying the molar concentration for each species of modifier by a correction factor that (again, according to theory) accounts for the number of active species present in a specific modifier. These correction factors are determined for each individual hydrocarbon and modifier by what is, apparently, a complicated process of experimental trial and error; however, the process for determining these correction factors is not set out with specificity in the aforementioned patents nor any actual examples of the procedure presented. Within this same prior art, it is also taught that “when the reaction temperature is increased or decreased, the position of the selectivity curve for the modifier [moderator] shifts towards a higher value of Q or a lower value of Q, respectively, proportionally with the change in the reaction temperature.”

Similarly, U.S. Pat. Nos. 7,102,022 and 7,485,597 also teach that “deviations from the optimum selectivity which would result from a change in temperature may be reduced or even prevented by adjusting the value of Q proportionally with the change in catalyst temperature.”

These four disclosures mentioned above teach that Q must be adjusted in a linear fashion with temperature according to the relationship: Q₂=Q₁+B(T₂−T₁) where T is temperature, Q is the ratio of the total effective modifier to the total effective hydrocarbon, and B is the linear proportionality constant.

EP Patent No. 0 352 850 B1 teaches that “after the catalyst has ‘lined-out’ and normal operating conditions are reached,” that the “chlorohydrocarbon moderator [be] slowly increased over the run time at an average rate of increase of at least 0.5% per month during the operation of the catalyst, more preferably at an average rate of increase of at least 19% per month and even more preferably at an average rate of increase of at least 3% per month and yet even more preferably at an average rate of increase of at least 5% per month.” While EP 0 352 850 B1 prescribes the need to increase the concentration of the moderator in the feed with operation time, it fails to provide an approach for the critical element of maintaining optimum moderator concentration with temperature change.

U.S. Pat. No. 9,221,766 discloses a method for the epoxidation of an olefin that includes reacting a feed gas composition containing an olefin. oxygen, and a halocarbon moderator, having a first moderator concentration M₁, in the presence of an epoxidation catalyst at a first temperature T₁. increasing the first temperature to a second temperature T₂, and increasing the first moderator concentration to a second moderator concentration M₂; wherein. the second moderator concentration is defined by the following expression: M₂=M₁(1+r)^{T}₂^{-T}₁ wherein the temperature has the units of degrees Celsius, and r is a constant factor which is in the range of from 0.001% to 100%. The '766 patent correlates the change in moderator concentration with the change in temperature to keep the catalyst operating at peak efficiency in the oxidation of ethylene to ethylene oxide.

### SUMMARY

A moderator management process that can be employed during an epoxidation process is disclosed. The disclosed moderator management process provides optimum catalyst performance without having to rely on a trial and error process or by using elaborate equations as disclosed in the prior art. In the disclosed moderator management process, the optimum moderator concentrations can be determined by maintaining ΔEa from 30 kJ/mol to 300 kJ/mol. ΔEa is defined herein as the difference between activation energies for the reaction of halide removal from the surface of the epoxidation catalyst and the reaction of halide deposition on the surface of the epoxidation catalyst, at a given reaction temperature.

In one aspect of the present disclosure, a process for the epoxidation of an olefin is provided. In one embodiment, the process includes reacting a feed gas comprising an olefin, oxygen and a halide-containing moderator, having a first optimized moderator concentration, in the presence of an epoxidation catalyst, and at a first temperature. Next, the first temperature is increased to a second temperature, and thereafter, the first optimized moderator concentration is increased to a second optimized moderator concentration. In the present disclosure, the first and second optimized moderator concentrations are determined by maintaining ΔEa from 30 kJ/mol to 300 kJ/mol, more preferably from 50 kJ/mol to 120 kJ/mol, still more preferably from 70 kJ/mol to 90 kJ/mol, and even more preferably from 75 kJ/mol to 85 kJ/mol. In one embodiment, ΔEa is 80 kJ/mol.

### BRIEF DESCRIPTION OF THE DRAWINGS

FIG. 1 is an Arrhenius plot used to obtain the value of activation energy in Example 1.

### DETAILED DESCRIPTION

The present disclosure will now be described in greater detail. Numerous specific details are set forth, such as particular structures, components, materials, dimensions, processing steps, and techniques to provide an understanding of the various embodiments of the present disclosure. However, it will be appreciated by one of ordinary skill in the art that the various embodiments of the present disclosure can be practiced without these specific details. As used throughout the present disclosure, the term about generally indicates no more than ±10%, ±5%, ±2%, ±1% or ±0.5% from a number. When a range is expressed in the present disclosure as being from one number to another number (e.g., 20 to 40), the present disclose contemplates any numerical value that is within the range (i.e., 22, 24, 26, 28.5, 31, 33.5, 35, 37.7, 39 or 40) or in any amount that is bounded by any of the two values within the range (e.g., 28.5-35).

The terminology used herein is for the purpose of describing particular embodiments only and is not intended to be limiting of the present disclosure. As used herein, the singular forms a, an, and the are intended to include the plural forms as well, unless the context clearly indicates otherwise. It will be further understood that the terms comprises and/or comprising, when used in this disclosure, specify the presence of stated features, integers, steps, operations, elements, and/or components, but do not preclude the presence or addition of one or more other features, integers, steps, operations, elements, components, and/or groups thereof.

Throughout the present application, and when reference is made to elements within the Periodic Table Elements, those elements are expressed in the previous IUPAC form unless otherwise stated to the contrary. Switching from the IUPAC form to the CAS version and/or new notation is well within the knowledge of one skilled in the art and can be done readily by referring to most modern Periodic Table of Elements.

The present disclosure relates to an epoxidation process in which moderator management is employed to provide optimum catalyst performance at a given reaction temperature. In epoxidation processes where halide-containing moderators are employed, the optimum moderator concentration on the epoxidation catalyst is a strong function of temperature. When the temperature varies, the total moderator concentration needs to be varied to maintain a constant and optimum moderator concentration. The Applicants of this disclosure have discovered that when a ΔEa from 30 kJ/mole to 300 kJ/mol is maintained during moderator management, with ΔEa as defined above, the moderator concentration remains optimized through temperature changes. When ΔEa is greater than 300 kJ/mol or below 30 kJ/mol, the halide concentration (i.e., moderator concentration) will not be optimized, and the catalyst performance will be less than optimum. This moderator management process will be described in greater detail following a description of the epoxidation catalyst that can be employed in the present disclosure.

The epoxidation catalyst employed in the present disclosure is any silver-based supported catalyst which achieves a selectivity during normal ethylene oxide production that is greater than 85 mole percent, preferably greater than 87 mole percent, more preferably, greater than 90 mole percent. The catalyst includes a support (i.e., catalyst carrier). The support can be selected from a large number of solid, refractory supports that can be porous. The support can comprise materials such as alpha-alumina, charcoal, pumice, magnesia, zirconia, titania, kieselguhr, fuller's earth, silicon carbide, silica, silicon carbide, clays, artificial zeolites, natural zeolites, silicon dioxide and/or titanium dioxide, ceramics, and combination thereof. One preferred support comprises alpha-alumina, having a high purity of at least 95 wt. %, or more preferably, at least 98 wt. % alpha-alumina. The remaining components of the preferred support can include inorganic oxides other than alpha-alumina, such as silica, alkali metal oxides (e.g., sodium oxide), and trace amounts of other metal-containing or non-metal-containing additives or impurities.

The support is preferably porous and has a B.E.T. surface area of at most 20 m²/g, preferably from 0.1 to 10 m²/g, and more preferably from 0.5 to 3 m²/g. As used herein, the B.E.T. surface area is deemed to have been measured by the method as described in Brunauer, Emmet and Teller in J. Am. Chem. Soc. 60 (1938) 309-316. The support can have a mono-modal pore size distribution or a multi-modal pore size distribution. Regardless of the character of the support used, it is usually shaped into particles, chunks, pieces, pellets, rings, spheres, wagon wheels, cross-partitioned hollow cylinders, and the like, of a size suitable for employment in fixed-bed epoxidation reactors. Desirably, the support particles can have equivalent diameters in the range of from about 3 mm to about 12 mm and preferably in the range of from about 5 mm to about 10 mm, which are usually compatible with the internal diameters of the tubular reactors in which the catalyst is placed. Equivalent diameter is the diameter of a sphere having the same external surface (i.e., neglecting surface within the pores of the particle) to volume ratio as the support particles being employed.

In order to produce a catalyst for the oxidation of ethylene to ethylene oxide, a support having the above characteristics is then provided with a catalytically effective amount of silver on its surface. The catalyst is prepared by impregnating the support with a silver compound, complex, or salt dissolved in a suitable solvent sufficient to cause deposition of a silver-precursor compound onto the support. Preferably, an aqueous silver solution is used. After impregnation, the excess solution is removed from the impregnated support, and the impregnated support is heated to evaporate the solvent and to deposit the silver or silver compound on the support as is known in the art. Preferred catalysts prepared in accordance with this disclosure contain up to about 45% by weight of silver, expressed as metal, based on the total weight of the catalyst including the support. The silver is deposited upon the surface and throughout the pores of the porous refractory support. Silver contents, expressed as metal, of from about 1% to about 40% based on the total weight of the catalyst, are preferred while silver contents of from about 8% to about 35% are more preferred. The amount of silver deposited on the support or present on the support is that amount which is a catalytically effective amount of silver; i.e., an amount which economically catalyzes the reaction of ethylene and oxygen to produce ethylene oxide. As used herein, the term catalytically effective amount of silver refers to an amount of silver that provides a measurable conversion of ethylene and oxygen to ethylene oxide. Useful silver containing compounds which are silver precursors include but are not limited to silver oxalate, silver nitrate, silver oxide, silver carbonate, a silver carboxylate, silver citrate, silver phthalate, silver lactate, silver propionate, silver butyrate, and higher fatty acid salts and combinations thereof.

Also deposited on the support, either prior to, coincidentally with, or subsequent to the deposition of the silver is a promoting amount of a rhenium component, which can be a rhenium-containing compound or a rhenium-containing complex. The rhenium component can be present in an amount of from about 0.001 wt. % to about 1 wt. %, preferably from about 0.005 wt. % to about 0.5 wt. %, and more preferably from about 0.01 wt. % to about 0.1 wt. % based on the weight of the total catalyst including the support, expressed as the rhenium metal. While catalysts containing rhenium are preferred, the present disclosure is not limited to silver-containing epoxidation catalysts that contain rhenium as one of the promoters.

Also deposited on the support either prior to, coincidentally with, or subsequent to the deposition of the silver and rhenium are optional additional promoters, which are promoting amounts of an alkali metal or mixtures of two or more alkali metals, as well as optional promoting amounts of a Group IIA alkaline earth metal component or mixtures of two or more Group IIA alkaline earth metal components, and/or a transition metal component or mixtures of two or more transition metal components, all of which can be in the form of metal ions, metal compounds, metal complexes and/or metal salts dissolved in an appropriate solvent. The particular combination of silver, support, alkali metal promoters, rhenium component, and optional additional promoters of the instant disclosure will provide an improvement in one or more catalytic properties over the same combination of silver and support and none, or only one of the promoters.

As used herein the term promoting amount of a certain component of the catalyst refers to an amount of that component that works effectively to improve the catalytic performance of the catalyst when compared to a catalyst that does not contain that component. The exact concentrations employed, of course, will depend on, among other factors, the desired silver content, the nature of the support, the viscosity of the liquid, and solubility of the particular compound used to deliver the promoter into the impregnating solution. Catalytic performance includes the following properties: inter alia, operability (resistance to runaway), selectivity, activity, conversion, stability, and yield. It is understood by one skilled in the art that one or more of the individual catalytic-performance properties can be enhanced by a promoting amount of a certain component while other catalytic-performance properties can or cannot be enhanced or can even be diminished. It is further understood that different catalytic-performance properties can be enhanced at different operating conditions. For example, a catalyst having enhanced selectivity at one set of operating conditions can be operated at a different set of conditions wherein the improvement shows up in the activity rather than the selectivity. In the epoxidation process, it can be desirable to intentionally change the operating conditions to take advantage of certain catalytic-performance properties even at the expense of other catalytic-performance properties. The preferred operating conditions will depend upon, among other factors, feedstock costs, energy costs, by-product removal costs, and the like.

Suitable alkali metal promoters can be selected from lithium, sodium, potassium, rubidium, cesium or combinations thereof, with cesium being preferred in some embodiments, and combinations of cesium with other alkali metals, such as lithium, being especially preferred in another embodiment. The amount of alkali metal deposited or present on the support is to be a promoting amount. Preferably, the amount will range from about 10 ppm to about 3000 ppm, more preferably from about 15 ppm to about 2000 ppm, and even more preferably from about 20 ppm to about 1500 ppm, and as especially preferred from about 100 ppm to about 1200 ppm by weight of the total catalyst, measured as the metal.

Suitable alkaline earth metal promoters comprise elements from Group IIA of the Periodic Table of the Elements, which can be beryllium, magnesium, calcium, strontium, and barium or combinations thereof. Suitable transition metal promoters can comprise elements from Groups IVA, VA, VIA, VIIA and VIIIA of the Periodic Table of the Elements, and combinations thereof. Most preferably the transition metal comprises an element selected from Groups IVA, VA or VIA of the Periodic Table of the Elements. Preferred transition metals that can be present include molybdenum, tungsten, chromium, titanium, hafnium, zirconium, vanadium, tantalum, niobium, or combinations thereof.

The amount of alkaline earth metal promoter(s) and/or transition metal promoter(s) deposited on the support are promoting amounts. The transition metal promoter can typically be present in an amount of from about 0.1 micromoles per gram to about 10 micromoles per gram, preferably from about 0.2 micromoles per gram to about 5 micromoles per gram, and more preferably from about 0.5 micromoles per gram to about 4 micromoles per gram of total catalyst, expressed as the metal. The catalyst can further comprise a promoting amount of one or more sulfur compounds, one or more phosphorus compounds, one or more boron compounds, one or more halogen-containing compounds, or combinations thereof. In one embodiment, the catalyst includes from about 5 to about 200 ppm, preferably from about 10 to about 100 ppm sulfur.

The silver solution used to impregnate the support can also comprise an optional solvent or a complexing/solubilizing agent such as are known in the art. A wide variety of solvents or complexing/solubilizing agents can be employed to solubilize silver to the desired concentration in the impregnating medium. Useful complexing/solubilizing agents include amines, ammonia, oxalic acid, lactic acid and combinations thereof. Amines include alkyl-or alkylene-diamines having from 1 to 5 carbon atoms. In one preferred embodiment, the solution comprises an aqueous solution of silver oxalate and alkyldiamine, ethane-1,2-diamine. The complexing/solubilizing agent can be present in the impregnating solution in an amount of from about 0.1 to about 5.0 moles per mole of silver, preferably from about 0.2 to about 4.0 moles, and more preferably from about 0.3 to about 3.0 moles for each mole of silver.

When a solvent is used, it can be an organic solvent or water, and can be polar or substantially or totally non-polar. In general, the solvent should have sufficient solvating power to solubilize the solution components. At the same time, it is preferred that the solvent be chosen to avoid having an undue influence on or interaction with the solvated promoters. Examples of organic solvents include, but are not limited to, alcohols, in particular alkanols; glycols, in particular alkyl glycols; ketones; aldehydes; amines; tetrahydrofuran; nitrobenzene; nitrotoluene; glymes, in particular glyme, diglyme and tetraglyme; and the like. Organic-based solvents which have 1 to about 8 carbon atoms per molecule are preferred. Mixtures of several organic solvents or mixtures of organic solvent(s) with water can be used, provided that such mixed solvents function as desired herein.

The concentration of silver in the impregnating solution is typically in the range of from about 0.1% by weight up to the maximum solubility afforded by the particular solvent/solubilizing agent combination employed. It is generally very suitable to employ solutions containing from 0.5% to about 45% by weight of silver, with concentrations of from 5 to 35% by weight of silver being preferred.

Impregnation of the selected support is achieved using any of the conventional methods, for example, excess solution impregnation, incipient wetness impregnation, spray coating, etc. Typically, the support material is placed in contact with the silver-containing impregnation solution until a sufficient amount of the solution is adsorbed by the support. Preferably the quantity of the silver-containing impregnation solution used to impregnate the porous support is no more than is necessary to fill the pores of the support. A single impregnation or a series of impregnations, with or without intermediate drying, can be used, depending, in part, on the concentration of the silver component in the solution. Impregnation procedures are described in U.S. Pat. Nos. 4,761,394, 4,766,105, 4,908,343, 5,057,481, 5,187,140, 5,102,848, 5,011,807, 5,099,041 and 5,407,888 and are herein incorporated by reference. Known prior procedures of pre-deposition, co-deposition, and post-deposition of various promoters can also be employed.

After impregnation of the support with the silver-containing compound, (i.e., a silver precursor), rhenium component, alkali metal component, and optional additional promoters, the impregnated support is calcined for a time sufficient to convert the silver-containing compound to an active silver species and to remove the volatile components from the impregnated support to generate the catalyst. The calcination can be accomplished by heating the impregnated support, preferably at a gradual rate, to a maximum temperature in the range of from about 200° C. to about 600° C., preferably from about 200° C. to about 500° C., and more preferably from about 200° C. to about 450° C., at a pressure in the range of from 0.5 bar to 35 bar. In general, the higher the maximum temperature, the shorter the required heating period. A wide range of heating periods have been suggested in the art; e.g., U.S. Pat. No. 3,563,914 suggests heating for less than 300 seconds, and U.S. Pat. No. 3,702,259 discloses heating from 2 to 8 hours at a temperature of from 100° C. to 375° C., usually for duration of from about 0.5 to about 8 hours. However, it is only important that the heating time be correlated with the temperature such that substantially all of the contained silver is converted to the active silver species. Continuous or step-wise heating can be used for this purpose.

During calcination, the impregnated support can be exposed to a gas atmosphere comprising an inert gas or a mixture of an inert gas with from about 10 ppm to 21% by volume of an oxygen-containing oxidizing component. For purposes of this disclosure, an inert gas is defined as a gas that does not substantially react with the catalyst or catalyst precursor under the conditions chosen for the calcination. Non-limiting examples include nitrogen, argon, krypton, helium, and combinations thereof, with the preferred inert gas being nitrogen. Non-limiting examples of the oxygen-containing oxidizing component include molecular oxygen (O₂), NO, NO₂, N₂O, N₂O₃, N₂O₄, or N₂O₅, or a substance capable of forming NO, NO₂, N₂O, N₂O₃, N₂O₄, or N₂O₅ under the calcination conditions, or combinations thereof, and optionally comprising SO₃, SO₂, trimethyl phosphite or combinations thereof. Of these, molecular oxygen is a useful embodiment, and a combination of O₂ with NO or NO₂ is another useful embodiment. In a useful embodiment, the atmosphere comprises from about 10 ppm to about 1% by volume of an oxygen-containing oxidizing component. In another useful embodiment, the atmosphere comprises from about 50 ppm to about 500 ppm of an oxygen-containing oxidizing component.

After calcination, the catalyst is loaded into reactor tubes of an epoxidation reactor utilizing conventional loading methods well known to those skilled in the art to provide a catalyst bed containing the catalyst. The catalyst bed can have packing densities that are suitable for the size of the reactor tubes that are being used. After loading the catalyst into the reactor tubes, an inert gas such as nitrogen, can be swept over the catalyst bed to remove any unwanted contaminates from the surface of the catalyst in the packed bed.

Next, any start-up process or moderator soak (or moderator break-through) process can be employed. In one example, the start-up process can include initiating an epoxidation reaction using an initial feed gas composition of olefin such as, for example, ethylene, and oxygen, adding a moderator to the initial feed gas composition, and increasing ethylene oxide production to the desired level.

After start-up or moderator soak (or moderator break-through), the epoxidation process can be carried out by continuously contacting oxygen with an olefin, preferably ethylene, in the presence of the previously described epoxidation catalyst; the olefin and oxygen are components of a feed gas (or reaction) composition that is added to the reactor during the epoxidation process. Oxygen may be supplied to the reactor in substantially pure molecular form or in a mixture such as air. Molecular oxygen employed as a reactant may be obtained from conventional sources. Feed-gas compositions that can be used during the epoxidation process can contain from about 0.5% to about 45% ethylene and from about 3% to about 15% oxygen, with the balance being mostly methane but also comprising comparatively inert materials, including such substances as carbon dioxide, water, inert gases, other hydrocarbons, and one or more reaction modifiers such as organic halides (herein, the moderator), inorganic halides, nitrogen oxides, phosphorus compounds, sulfur compounds and mixtures thereof. Non-limiting examples of inert gases include nitrogen, argon, helium and mixtures thereof. Non-limiting examples of the other hydrocarbons include ethane, propane, and mixtures thereof. Carbon dioxide and water are byproducts of the epoxidation process. Both have adverse effects on the catalyst performance, so the concentrations of these components are usually kept at a minimum.

As described, the feed gas also includes one or more halide-containing moderators. Non-limiting examples of halide-containing moderators include organic halides such as C₁ to C₈ halohydrocarbons. Preferably, the halide-containing moderator is methyl chloride, ethyl chloride, ethylene dichloride, ethylene dibromide, vinyl chloride or mixtures thereof. Most preferred halide-containing moderators are ethyl chloride and ethylene dichloride. During the epoxidation process, the concentration of these halide-containing moderators in the feed gas needs to be adjusted to maintain optimum catalyst performance.

During the epoxidation process, the halide, e.g., chloride, from the halide-containing moderator described above is adsorbed on the surface of the epoxidation catalyst. The concentration of halide adsorbed on the catalyst surface dynamically depends upon the relative rates of reactions that add the halide to the catalyst surface and competing reactions that remove the halide from the catalyst surface. Though not wishing to be bound by theory, it is believed that in this dynamic process, the halide, e.g., chloride, is added to the catalyst's surface by oxydechlorination of chlorinated hydrocarbon moderators in the feed gas to the reactor, and chloride is removed from the surface of the catalyst by oxychlorination of hydrocarbons in the feed gas to the reactor. Regardless of the exact reaction mechanisms involved, one reaction sequence works to add halide to the surface of the catalyst (herein, the halide addition reaction, or the chloride addition reaction), and one reaction sequence works to remove halide from the surface of the catalyst (herein, the halide removal reaction, or the chloride removal reaction). There is a concentration of halide adsorbed on the catalyst surface that gives optimum catalyst performance, and this is facilitated by having a corresponding optimum concentration of the halide-containing moderator in the feed gas to the reactor. Here, optimum catalyst performance is defined as the combination of maximum olefin oxide selectivity and lowest reactor coolant temperature. When rhenium is present in the epoxidation catalyst, the optimum halide-containing moderator concentration range can be very narrow. In the present disclosure, optimized halide-containing moderator concentration is achieved when the dynamics of the two competitive reactions mentioned above-the halide addition reaction and the halide removal reaction-are in proper balance.

Specifically, the moderator management process of the present disclosure includes reacting a feed-gas composition that includes an olefin, oxygen, and a halide-containing moderator (all as defined above), having a first optimized moderator concentration in the presence of an epoxidation catalyst (as defined above) and at a first temperature. The concentration of olefin and oxygen in the feed gas composition are as described above. In embodiments of the present disclosure, the concentrations of olefin and oxygen in the feed gas remain constant. In the present disclosure, the first temperature can be a temperature within a range from 180° C. to 260° C., with a temperature within a range from 220° C. to 240° C. being more preferred for the first temperature. Here, the first temperature is the reactor coolant temperature, which can be measured utilizing techniques well known to those skilled in the art.

Next, the first temperature is increased to a second temperature. In the present disclosure, the second temperature can be a temperature within a range from 220° C. to 320° C., with a temperature within a range from 240° C. to 280° C. being more preferred for the second temperature. Here, the second temperature is again the reactor coolant temperature, which can be measured by techniques well known to those skilled in the art.

In the present disclosure, the first temperature can be any temperature within the epoxidation process including a lowest temperature (e.g., 180° C.) of the epoxidation process in which epoxidation has just been initiated, and the second temperature is any temperature in the epoxidation process that is higher than the first temperature. The difference between the first temperature and the second temperature can be in a range from 30° C. to 80° C., with a difference between the first temperature and the second temperature from 40° C. to 60° C. being more preferred. Generally, the epoxidation process in which moderator management is implemented occurs between a temperature of about 180° C. to about 320° C., and moderator management can be used within this entire temperature range.

In the present disclosure, the increasing of the first temperature to the second temperature is performed when the catalyst performance (selectivity and/or activity) begins to decline. Notably, the increasing of the first temperature to the second temperature is performed when a drop of catalyst selectivity is observed at the first temperature. In some embodiments, the increasing of the first temperature to the second temperature is performed when the selectivity of the catalyst at the first temperatures drops below the optimum selectivity of the epoxidation catalyst. For example, the increasing to the second temperature can occur when the selectivity is less than 85 mole percent, preferably less than 87 mole percent, more preferably, less than 90 mole percent.

At the second temperature, the first optimized moderator concentration is increased to a second optimized moderator concentration to again achieve the optimum selectivity of the catalyst at the second temperature. In an exemplary embodiment, the first moderator concentration can be within a range from about 0.2 ppm to about 10 ppm, and the second moderator concentration can be in a range from about 0.5 ppm to about 20 ppm.

In the present disclosure, the first and second optimized moderator concentrations are determined by maintaining ΔEa from 30 kJ/mol to 300 kJ/mol, more preferably from 50 kJ/mol to 120 kJ/mol, still more preferably from 70 kJ/mol to 90 kJ/mol, and even more preferably from 75 kJ/mol to 85 kJ/mol (including any numerical value in these ranges such as, and for the range from 70 kJ/mol to 90 kJ/mol, 70.5, 71, 71.5, 72, 72.5, 73, 73.5 . . . 85, 85.5, 86, 86.5, 87, 87.5, 88, 88.5, 89, 89.5 and 89.9)), wherein ΔEa is as defined above. In some embodiments of the present disclosure, the value of ΔEa is about 80 kJ/mole (including 80 kJ/mole itself).

In the present disclosure, ΔEa can be determined by the equation r=ke^{-Ea/RT}, where r is the net rate of halide, e.g., chloride, deposition on the catalyst surface, k is the rate constant, R is the ideal gas constant, and T is the temperature in Kelvin. The value of r is assumed to be linearly proportional to the concentration of chloride-containing moderator in the reactor feed. Because the rate of the chloride removal reaction increases faster with temperature than the rate of the chloride addition reaction, the concentration of the chloride-containing moderator in the feed must be increased with reaction temperature to maintain the optimum concentration of chloride on the catalyst surface. To maintain the optimum concentration of the chloride-containing moderator in the feed over this change in reaction temperatures, the precise amount by which the first concentration of the chloride-containing moderator must be increased to the second concentration of the chloride-containing moderator is determined by the ΔEa value, which must be between the ranges that are recited herein, i.e., from 30 kJ/mol to 300 kJ/mol, more preferably from 50 kJ/mol to 120 kJ/mol, still more preferably from 70 kJ/mol to 90 kJ/mol, and even more preferably from 75 kJ/mol to 85 kJ/mol.

The above moderator management process can continue throughout the entire epoxidation process and can be used to adjust the moderator concentration at different temperatures during the epoxidation process by monitoring ΔEa. This moderator management can be performed by a reactor operator, or it can be automated, using a computer program to determine and automate the adjustment of the optimized moderator concentration for a given reaction temperature.

A usual method for the ethylene epoxidation process comprises the vapor-phase oxidation of ethylene with molecular oxygen, in the presence of an epoxidation catalyst in a fixed-bed tubular reactor. Conventional, commercial fixed-bed ethylene-oxide reactors are typically in the form of a plurality of parallel elongated tubes (in a suitable shell) approximately 0.7 to 2.7 inches O.D. and 0.5 to 2.5 inches I.D. and 15-45 feet long filled with catalyst and some packing materials at the top and bottom of the tubes. Typical operating conditions for the ethylene epoxidation process involve temperatures of the process in the range of from about 180° C. to about 330° C., and preferably, about 200° C. to about 325° C., and more preferably from about 220° C. to about 270° C. The operating pressure may vary from about atmospheric pressure to about 30 atmospheres, depending on the mass velocity and productivity desired. Higher pressures may be employed within the scope of the invention. Residence times in commercial-scale reactors are generally on the order of about 0.3-3 seconds.

The resulting ethylene oxide is separated and recovered from the reaction products using conventional methods. For the present disclosure, the ethylene epoxidation process may include a gas recycle wherein substantially all of the reactor effluent is readmitted to the reactor inlet after substantially or partially removing the ethylene oxide product and the byproducts. In the recycle mode, carbon dioxide concentrations in the gas inlet to the reactor may be, for example, from about 0.1 to 10 volume percent.

The previously described moderator management process is particularly suitable for oxidation of ethylene with molecular oxygen to ethylene oxide. The conditions for carrying out such an oxidation reaction in the presence of the catalysts broadly comprise those described in the prior art. This applies to suitable temperatures, pressures, residence times, diluent materials, moderating agents, and recycle operations, or applying successive conversions in different reactors to increase the yields of ethylene oxide. For purposes of illustration only, the following are conditions that are often used in current commercial ethylene oxide reactor units: a gas hourly space velocity of 1500-10,000 h⁻¹, a reactor inlet pressure of 150-400 psig, a coolant temperature of 180° C.-315° C., an oxygen conversion level of 10-60%, and an EO production rate (work rate) of 50-500 kg EO/m³ catalyst·hour. The feed composition at the reactor inlet may typically comprises 1-40% ethylene, 3-12% O₂, 0.3-40% CO₂, 0-3% ethane, 0.3-20 ppmv total concentration of organic chloride moderator(s), and the balance of the feed being comprised of argon, methane, nitrogen or mixtures thereof.

Examples have been set forth below for the purpose of illustrating the present disclosure. The scope of the present disclosure is not limited to the examples set forth herein. The general procedure for determining ΔEa is as follows. With a reactor feed gas containing a certain concentration of ethyl chloride moderator, both the chloride addition, and the chloride removal reactions work to add/remove Cl from the catalyst surface, respectively, and the difference in activation energies between these two reactions ΔEa can be experimentally determined by using the equation p_{EC}=ke^{-ΔEa/RT}, wherein p_{EC} is the molar concentration of ethyl chloride moderator in the reactor feed, with assumptions (1) the catalyst surface chloride is optimized for the optimal performance, (2) the concentration of each component of the feed is constant, except ethyl chloride that varies with varying reactor temperature, (3) ethyl chloride is the only chloride-containing moderator in the feed, and (4) the chloride addition reaction is first order in ethyl chloride.

### EXAMPLE 1

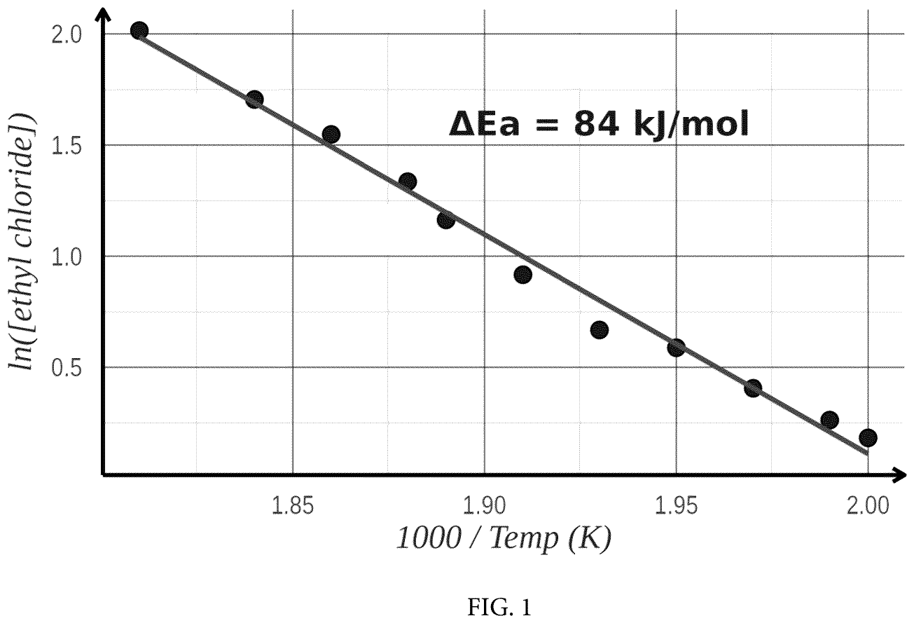

Example 1 illustrates the impact of chloride concentration on the catalyst performance as a function of temperature of a highly selective catalyst (Catalyst A). Catalyst A is a highly selective catalyst having a silver content of about 16.5 weight percent on an alpha alumina carrier with promoters of Cs, Re, W, Li, and S. Each promoter is present in a promoting amount as defined above. The catalyst was tested in a feed mixture comprising 30% C₂H₄, 7% O₂, 1% CO₂, an optimized amount of ethyl chloride, and nitrogen balance at a gas hourly space velocity of 4475 h⁻¹. To achieve the optimal performance of the catalyst, the concentration of the ethyl chloride moderator in the reactor feed was varied with temperature from 225° C. to 280° C. Table 1 shows experimental data for the optimum ethyl chloride concentration measured at different process temperatures. In Table 1, Ln (EC) denotes the log of ethyl chloride concentration present at the given temperature. The difference of the activation energies between the chloride removal and chloride addition reactions, ΔEa, was determined to be 84 kJ/mol, using the Arrhenius plot shown in FIG. 1 that is based on the equation p_{EC}=ke^{-Ea/RT}, as mentioned above.

TABLE 1
Optimum Ethyl Chloride Concentrations Measured
at Different Process Temperatures

| Process | Process | EC | | |
|---|---|---|---|---|
| Temperature | Temperature | Concentration | | |
| (° C.) | (Kelvin; K) | (ppm) | 1000/T(K) | Ln (EC) |
| 227 | 500 | 1.2 | 2.00 | 0.1823216 |
| 230 | 503 | 1.3 | 1.99 | 0.2623643 |
| 235 | 508 | 1.5 | 1.97 | 0.4054651 |
| 240 | 513 | 1.8 | 1.95 | 0.5877867 |
| 245 | 518 | 1.95 | 1.93 | 0.6678294 |
| 250 | 523 | 2.5 | 1.91 | 0.9162907 |
| 255 | 528 | 3.2 | 1.89 | 1.1631508 |
| 260 | 533 | 3.8 | 1.88 | 1.3350011 |
| 265 | 538 | 4.7 | 1.86 | 1.5475625 |
| 270 | 543 | 5.5 | 1.84 | 1.7047481 |
| 278 | 551 | 7.5 | 1.81 | 2.014903 |

### EXAMPLE 2

Example 2 illustrates the impact of chloride moderator concentration on catalyst selectivity as a function of temperature for the same catalyst as Example 1. The catalyst was tested in a feed mixture comprising 30% C₂H₄, 7.5% O₂, 1% CO₂, an optimized amount of ethyl chloride moderator, and methane balance at a gas hourly space velocity of 2830 h⁻¹. To maintain optimal catalyst performance, the concentration of the ethyl chloride moderator was varied with the temperature of the process in the range from 235° C. to 250° C. The difference in activation energies between the chloride removal and chloride addition reactions, ΔEa, was determined to be 85 kJ/mol.

### EXAMPLE 3

Example 3 illustrates the impact of chloride moderator concentration on catalyst selectivity as a function of temperature for the same catalyst as Example 1. The catalyst was tested in a feed mixture comprising 34.6% C₂H₄, 7.8% O₂, 0.3% CO₂, an optimized amount of ethyl chloride moderator, and methane balance at a gas hourly space velocity of 4480 h⁻¹. To achieve the optimal performance of the catalyst, the concentration of the optimal ethyl chloride varied with temperature of the process from 223° C. to 240° C. The difference in activation energies between the chloride removal and chloride addition reactions was determined to be 81 kJ/mol.

While the present disclosure has been particularly shown and described with respect to preferred embodiments thereof, it will be understood by those skilled in the art that the foregoing and other changes in forms and details can be made without departing from the spirit and scope of the present disclosure. It is therefore intended that the present disclosure is not limited to the exact forms and details described and illustrated, but fall within the scope of the appended claims.

What is claimed is:

1. A process for epoxidation of an olefin, the process comprising:
reacting a feed gas composition comprising an olefin, oxygen and a halide-containing moderator having a first optimized moderator concentration in the presence of an epoxidation catalyst and at a first temperature;
increasing the first temperature to a second temperature; and
increasing, at the second temperature, the first optimized moderator concentration to a second optimized moderator concentration, wherein the first and second optimized moderator concentrations are determined by maintaining a ΔEa from 30 kJ/mol to 300 kJ/mol, wherein ΔEa is the difference in activation energies between the reaction to remove halide from the surface of the epoxidation catalyst and the reaction to deposit halide on the surface of the epoxidation catalyst.

2. The process of claim 1, wherein the ΔEa is from 50 kJ/mol to 120 kJ/mol.

3. The process of claim 1, wherein the ΔEa is from 70 kJ/mole to 90 kJ/mole.

4. The process of claim 1, wherein the ΔEa is from 75 kJ/mole to 85 kJ/mole.

5. The process of claim 1, wherein the ΔEa is about 80 kJ/mole.

6. The process of claim 1, wherein the epoxidation catalyst is a silver-based catalyst having a selectivity of greater than 85 mole percent.

7. The process of claim 6, wherein the silver-based catalyst includes silver in an amount up to 45% by weight.

8. The process of claim 7, wherein the silver is supported on an alpha alumina-containing carrier having a surface area of at most 20 m²/gm.

9. The process of claim 7, wherein the silver-based catalyst further comprises a promoting amount of rhenium.

10. The process of claim 9, wherein the promoting amount of rhenium is from about 0.01 wt. % to about 1 wt. %.

11. The process of claim 9, wherein the silver-based catalyst further comprises a promoting amount of at least one alkali metal.

12. The process of claim 11, wherein the at least one alkali metal is a combination of lithium and cesium.

13. The process of claim 11, wherein the silver-based catalyst further comprises a promoting amount of at least one transition metal selected from Mo, W, Cr, Ti, Hf, Zr, V, Ta and Nb.

14. The process of claim 6, wherein the silver-based catalyst comprises an alpha aluminum carrier, up to 45 percent by weight silver, and promoting amounts of rhenium, lithium, cesium, tungsten, and sulfur.

15. The process of claim 1, wherein the olefin comprises ethylene.

16. The process of claim 1, wherein the first temperature is within a range from about 180° C. to 260° C., and the second temperature is within a range from about 220° C. to 320° C.

17. The process of claim 1, wherein the first temperature is within a range from about 220° C. to 240° C., and the second temperature is within a range from about 240° C. to 280° C.

18. The process of claim 1, wherein the first optimized moderator concentration is from about 0.2 ppm to about 10 ppm, and the second optimized moderator concentration is from about 0.5 ppm to about 20 ppm.

19. The process of claim 1, wherein the feed gas composition comprises from about 5% to 40% ethylene and from about 3% to about 15% oxygen.

20. The process of claim 1, wherein the halide-containing moderator is a chlorohydrocarbon or a mixture of one or more chlorohydrocarbons selected from the group consisting of methyl chloride, ethyl chloride, ethylene dichloride, and vinyl chloride.
